(19) United States
(12) Patent Application Publication
(10) Pub. No.: US 2012/0129942 A1
(43) Pub. Date: May 24, 2012

(54) COMPOSITIONS AND METHODS FOR TREATING FUNGAL INFECTION OF THE NAIL

(75) Inventor: Ake Lindahl, Malmo (SE)

(73) Assignee: MOBERG DERMA AB, Bromma (SE)

(21) Appl. No.: 13/383,510

(22) PCT Filed: Aug. 13, 2010
(86) PCT No.: PCT/SE2010/050886

§ 371 Date: Jan. 11, 2012

Related U.S. Application Data
(60) Provisional application No. 61/233,557, filed on Aug. 13, 2009.

(51) Int. Cl. A61K 31/137; A61K 31/17; A61P 31/10

(52) U.S. Cl. 514/588; 514/650

(57) ABSTRACT

A stable antifungal composition for topical application on a nail comprising a diol component, an organic acid component, a volatile vehicle, an antifungal agent and a keratolytic agent; the active compound and the keratolytic agent are soluble in the composition in the absence of said volatile vehicle, and wherein at least one in the group selected from the antifungal agent and the keratolytic agent is present in solid state in the composition in the presence of said volatile vehicle. Preferred ingredients include propylene glycol, lactic acid, ethyl acetate, urea and terbinafine or naftifine.

### TECHNICAL FIELD

The present invention relates generally to compositions and methods for the treatment of fungal nail infections, in particular onychomycosis.

### BACKGROUND

Fungal infection of the nail, in particular onychomycosis, is the most common disease of the nail and affects as much as 6-8% of the adult population. It manifests itself by opaque, white, brittle, thick, and friable nails caused by the invasion of fungi.

The search for an efficient treatment of fungal infections of keratinous structures such as the nail has been subject to numerous efforts but so far no satisfactory solution is at hand. There is a general agreement that if sufficient amounts of a potent antifungal compound can be distributed throughout the nail and in the nail bed, the infection will be cured and destruction of the nail will end.

Although many promising attempts have been made with nail penetration antifungal agents, many one of these products have showed little effect in clinical testing. One reason is that the minimum inhibitory concentration (MIC) value of antifungals for fungi feeding on a strict keratin diet has values that are many times higher than the MIC values calculated at in vitr conditions. Therefore, previous estimates regarding the extent of drug penetration that was needed were much too low for the treatment to have an effect. The result of the treatment of fungal infections of the nail, such as onychomycosis, still depends on the success in generating a sufficiently high penetration of an antifungal compound.

Further, the treatment times using existing therapies have been long, up to more than one year, resulting in poor adherence.

Another problem in the field is the stability of the active component. Several of the antifungal agents are not stable, which results in the active compound breaking down over time. This limits the shelf life of the product.

A major problem associated with topical administration of antifungal drugs to the nail is the barrier function of the keratinous layer. One way is to break up the structure-forming component of the nail: keratin. This can be done by keratolytic compounds exemplified by acetylcysteine, thioglycollic acid and urea. However, these compounds may further contribute to the breakdown of the active substance. One example of such a component is urea, which decreases the stability of terbinafine.

### DEFINITIONS

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation±10% of the stated value, where applicable.

The term “film forming agent” is used to denote a compound or a mixture of compounds that is pharmaceutically acceptable and which increases the viscosity of a pharmaceutical composition intended to be applied topically.

In addition to the above, the expression “in the solid state” is used to indicate that a compound is present in precipitated form, as opposed to dissolved, in the composition. That a compound is present in solid state can be confirmed by the naked eye where the presence of visible particles or aggregates confirms the solid state.

As used herein, unless stated otherwise, the amounts (of components) in percent refer to percent by weight and are based on the total weight of the composition.

### SUMMARY

It is an object of the invention to address at least some of the issues outlined above. It would be desirable to increase the penetration of antifungal compositions. It would also be desirable to increase the stability and thereby the shelf-life of antifungal compositions. These and other objects are addressed by a composition, method, and use according to the attached claims, incorporated herein by reference.

The present inventors make available an antifungal composition for topical application on a nail, said composition comprising a diol component, an organic acid component, a volatile vehicle, an antifungal agent; and one or more keratolytic agent; wherein effective amounts of the active compound and said keratolytic agent are soluble in the composition in the absence of said volatile vehicle, and wherein at least one in the group selected from the antifungal agent and the keratolytic agent is present in the solid state in the composition in the presence of said volatile vehicle.

The inventors have surprisingly found that this composition is capable of delivering an antifungal component efficiently into the nail, and simultaneously exhibits improved stability and thereby longer shelf-life.

### DETAILED DESCRIPTION

The compositions according to the invention are intended to be applied topically to the nail of a patient suffering from a fungal infection of the nail, for example onychomycosis.

The volatile vehicle is chosen so that at least one of the antifungal agent and the keratolytic agent are precipitated in the composition in the presence of the volatile vehicle but so that the thus precipitated agents dissolve in the absence of the volatile vehicle, such as, for example, when the volatile vehicle has evaporated. Thus, during storage, at least one of the antifungal agent and the keratolytic agent are at least partly in the solid state. In the solid state, these components are less prone to undergo chemical reactions and degradation than in the dissolved state and are thus more stable. This is confirmed by stability tests presented in the examples.

Shortly after application, the volatile vehicle evaporates leaving the other compounds of the composition on the nail. Because the active compound and the keratolytic agent are soluble in the composition in the absence of the volatile vehicle, the active compound and the keratolytic agent dissolves upon the evaporation of the volatile vehicle. As the active compound and the keratolytic agent are released, they are redissolved in the other components of the composition, preferably within minutes, for example within 5 minutes.

The volatile vehicle is chosen so that it evaporates within 5 minutes, more preferably within 3 minutes after application in room temperature (18°-25° C.). A volatile vehicle with a vapor pressure of at least 2 kPa at 20° C. can be used.

Suitable volatile vehicles are generated from polar fluids such as esters, alcohols, ketones and saturated hydrocarbons with a high vapor pressure (greater than about 2 kPa at 20° C.). Vapor pressures of such volatile vehicles can be found, for example, in the CRC Handbook of Chemistry and Physics, 75^{th} edition (Vapor pressure of organic compounds), incorporated herein by reference. Examples of suitable volatile vehicles are ethyl acetate, butyl acetate, methyl acetate, isopropanol (isopropyl alcohol), ethanol, acetone, methyl ethyl ketone and methyl isobutyl ketone. The volatile vehicle is preferably chosen from ethyl acetate, butyl acetate and mixtures of these.

In one embodiment the mixture of ethyl acetate and butyl acetate is such that the composition comprises from about 30% to about 90% of ethyl acetate and from about 5% to about 60% of butyl acetate, based on the total weight of the composition. In a preferred embodiment the mixture of ethyl acetate and butyl acetate is such that the composition comprises from about 50% to about 70% of ethyl acetate and from about 20% to about 35% of butyl acetate, based on the total weight of the composition. In the most preferred embodiment the mixture of ethyl acetate and butyl acetate is such that the composition comprises from about 55% to about 65% of ethyl acetate and from about 22% to about 28% of butyl acetate, based on the total weight of the composition.

Other suitable volatile vehicles have vapor pressure at 20° C. that is equal or greater to one or more of the above mentioned compounds, and which demonstrate an equal or lower ability to dissolve an allylamine or other antifungal compound and urea or a another keratolytic agent.

Non-limiting examples of suitable volatile vehicles for compositions that comprise terbinafine and/or naftifine and, optionally, urea are volatile vehicles that comprise ethyl acetate or mixtures of ethyl acetate and butyl acetate resulting in a vehicle that evaporates within 5 minutes at 20° C.

The preferred amount of volatile vehicle is from about 70 to about 99%, more preferably from about 75% to about 96%, most preferably from about 78% to about 95%, based on the total weight of the composition.

The diol component and the organic acid component are present in an amount to provide penetration of the antifungal component through the nail in a pharmaceutically effective amount.

The diol component comprises at least one diol. Non-limiting examples of the diol component are ethylene glycol, propylene glycol, butanediol, pentanediol (for example 1,5-pentane diol), hexanediol, and mixtures thereof. If desired, the diol component may be a mixture of diols such as a mixture of propylene glycol and another diol, such as 1,5-pentanediol. A preferred diol is propylene glycol.

Suitable concentration ranges of the diol is from about 1% to about 20%, more preferably from about 3% to about 10%, even more preferably from about 6% to about 8%.

The organic acid component comprises, or consists essentially of, or consists of, a C₁₋₁₀ carboxylic acid or a solution thereof. Examples of C₁₋₁₀ carboxylic acid include any one or more of saturated or unsaturated, straight or branched aliphatic mono-, di- and polycarboxylic acids having 1, 2, 3, 4, 5, 6, 7, 8, 9 or 10 carbon atoms, araliphatic or aromatic dicarboxylic acids, oxy and hydroxyl carboxylic acids (e.g. alpha-hydroxy acids) having 1, 2, 3, 4, 5, 6, 7 or 8 carbon atoms. Examples of suitable organic acid components include one or more of formic acid, acetic acid, propionic acid, butyric acid, valeric acid, caproic acid, capryic acid, capric acid, sorbic acid, oxalic acid, citric acid, malonic acid, fumaric acid, succinic acid, glutaric acid, apidic acid, pimelic acid, oxalacetic acid, phtalic acid, malic acid, tartaric acid, tartronic acid, hydrobutyric acid, hydroxypropionic acid and pyruvic acid. A preferred organic acid is lactic acid.

Suitable concentration ranges of the organic acid component is from about 0.1% to about 4%, more preferably from about 0.3% to about 1.25%, even more preferably from about 0.8% to about 1.2%.

Suitable concentration ratios of the organic acid component and the diol are from 1:20 to 1:2, more preferably from 1:10 to 1:4.

The total combined concentration of the diol and the organic acid in the formulation is preferably from about 1% to about 50%, more preferably from about 2% to about 25%, and most preferably from about 4% to about 15%, based on the total weight of the composition.

The relation between organic acid and diol are preferably from about 1:20 to about 1:1, preferably from about 1:15 to about 1:2 and more preferably from about 1:12 to about 1:5, based on the total weight of the composition.

The diol component and the organic acid component are both characterized by low vapor pressure which results in an increase of their relative content in the evaporated formulation, that is, when the volatile vehicle has evaporated after application on the nail. The diol component and the organic acid component are chosen so that the antifungal agent and the keratolytic agent are readily dissolved in these in the absence of the volatile vehicle. Preferably the antifungal agent and the keratolytic dissolves in the diol component and the organic acid component within 5 minutes.

The antifungal agent is present in a pharmaceutically effective amount, which amount may vary depending upon the particular antifungal component(s) selected. Based on the disclosure herein, one of ordinary skill in the art will easily be able to select suitable amounts of antifungal component(s).

Preferred concentrations of the antifungal agent are about 0.01% to about 10%, more preferably from about 0.2% to about 5%, more preferably from about 0.75% to about 2.5%, more preferably from about 0.8% to about 1.2%, based on the total weight of the composition.

Examples of suitable antifungal agents include imidazoles, such as miconazole, ketoconazole, econazole, bifonazole, butoconazole, fenticonazole, isoconazole, oxiconazole, sertaconazole, sulconazole, and tioconazole; triazoles, such as fluconazole, itraconazole, isavuconazole, ravuconazole, posaconazole, voriconazole, and terconazole; thiazoles, such as abafungin; allylamines, such as terbinafine, amorolfine, naftifine, and butenafine; and echinocandins, such as anidulafungin, caspofungin, and micafungin, or mixtures of these.

Allylamine antifungal agents, in particular terbinafine and naftifine, are preferred antifungal agents of the present invention. These inhibit the growth of fungi by blocking the enzyme squalene epoxidase, a key enzyme in fungal ergosterol biosynthesis. Examples of suitable allylamines antifungal agents include an allylamine antifungal agent selected from the group consisting of amorolfine, butenafine, terbinafine and naftifine and mixtures of any two or more thereof. These are non-limiting examples of allylamine antifungal agents.

In addition, the composition comprises a pharmaceutically acceptable keratolytic agent, which enhances the penetration of the antifungal agent through the nail. Examples of the keratolytic agents include urea, one or more sulphur-containing amino acids, and mixtures thereof, with urea being the preferred keratolytic agent of the present invention.

Examples of suitable sulphur-containing amino acids include cysteine, methionine, N-acetyl cysteine, homocysteine, methyl cysteine, ethyl cysteine, N-carbomyl cysteine, glutathione, cysteamine or derivatives thereof.

A suitable concentration of the keratolytic agent is from about 0.3% to about 20%, more preferably from about 0.75% to about 2.5% and most preferably from about 1.8% to about 2.2%, based on the total weight of the composition.

In one embodiment the composition comprises from about 1% to about 20% of a diol, from about 0.1% to about 4% of an organic acid, from about 0.2% to about 5% of an antifungal agent, from about 0.3% to about 20% of a keratolytic agent, and from about 70% to about 99% of a volatile vehicle.

In one embodiment the composition comprises from about 3% to about 10% of a diol, from about 0.3% to about 1.25% of an organic acid, from about 0.75% to about 1.5% of an antifungal agent, from about 0.75% to about 2.5% of a keratolytic agent, and from about 75% to about 96% of a volatile vehicle.

In one embodiment the composition comprises from about 6% to about 8% of a diol, from about 0.8% to about 1.2% of an organic acid, from about 0.8% to about 1.2% of an antifungal agent, from about 1.8% to about 2.2% of a keratolytic agent, and from about 78% to about 95% of a volatile vehicle.

Furthermore, compounds that improve texture during administration and on the nail during treatment, such as a film forming agent, can be added to the composition according to embodiments of the invention. Suitable film forming properties results in an increased viscosity at administration which facilitates dosing and the formation of a film on the nail. This allows the product to stay at the surface of the nail to perform its effect. Preferably, according to one embodiment of the invention, the composition comprises a polymer having suitable film forming properties. Non-limiting examples of such compounds includes cellulose derivatives such as ethyl cellulose, cellulose acetate butyrate and polymethacrylates such as Eudragit. Suitable concentrations of a film-forming agent can be determined by a person skilled in the art.

If desired, the composition may further comprise a sequestration agent. Sequestration agents are believed to further enhance the penetration of an allylamine antifungal component trough nail tissue. Non-limiting examples of such sequestration agents include one or more of aminoacetic acids, phosphonates, phosphonic acids and mixtures of these. Sequestration agents can be metal complexing agents and thus, may form a complex with metals such as the alkali metals or alkaline earth metals. A preferred aminoacetic acid is ethylenediaminetetraacetic acid (EDTA). When included in the compositions, examples of suitable amounts of the sequestration agent include from about 0.01 to about 5% by weight, preferably from about 0.03% to about 0.5%.

In one embodiment, the composition further comprises a detergent. A non-limiting example of a suitable detergent is Tween 80. Suitable concentrations of detergent is from about 0.1% to about 5%, more preferably from about 0.5% to about 3%, even more preferably from about 0.7% to about 1.5%.

A preferred embodiment of the invention consists essentially of from about 6% to about 8% of a diol, from about 0.8% to about 1.2% of an organic acid, from about 1.8% to about 2.2% of a keratolytic agent, from about 0.8% to about 1.2% of an antifungal agent, from about 6% to about 10% of a film forming agent, from about 54% to about 60% of ethyl acetate, from about 22% to about 26% of butyl acetate, a detergent and a sequestering agent.

Another preferred embodiment of the invention consists essentially of about 7% of a diol, about 1% of an organic acid, about 2% of a keratolytic agent, about 1% of an antifungal agent, about 8% of a film forming agent, about 56% of ethyl acetate, about 24% of butyl acetate, a detergent and a sequestering agent.

It is preferred that embodiments demonstrate high nail penetration. This can be assessed by an in vitr method for nail penetration. For example, a Franz cell can be used to study the penetration through a membrane from a bovine hoof as described in the examples below.

In addition, the composition may contain buffering compounds in order to stabilize any acidic compounds in the formulation.

Other pharmaceutically acceptable carriers and excipients and agents such as stabilizers, penetration enhancers, and coloring may be added to the invented composition as desired, based on the knowledge of a skilled artisan.

A second main aspect of the invention makes available a method for treating a nail disease comprising administering the composition according to the invention to the nail of a patient. The nail disease is chosen among fungal infections of the nail, represented by, but not limited to, onychomycosis.

The compositions according to embodiments of the invention are preferably administered directly to the nail. For instance, the composition is administered on and around a human toe nail or finger nail affected by a fungal disease, such as onychomycosis. This may be performed by covering each affected nail from about twice per day to about once per week with a layer of the composition. The composition may also be applied to the edge of a nail. Administration of the composition by a suitable device such as a drop tip, a small brush or a spatula. Preferably, this is carried out at a temperature that allows the evaporation of the volatile vehicle within a suitable time, such as a few seconds or minutes. When the composition comprises a film forming agent, the patient allows the film to form.

A third aspect of the invention makes available a novel and improved composition for use in treating a nail disease, preferably onychomycosis.

A fourth aspect of the invention makes available the use of said composition for the manufacture of a medicament for treating a nail disease, preferably onychomycosis

While the claimed invention has been described in detail and with reference to specific embodiments thereof, it will be apparent to one of ordinary skill in the art that various changes and modifications can be made to the claimed invention without departing from the spirit and scope thereof.

### EXAMPLES

The invention will now be illustrated by means of examples, which are intended to show embodiments of the invention, but not to limit the scope of the inventive concept as set forth in the description and claims.

Precipitation and dissolution in the composition was determined as the presence or absence of particles observed by the naked eye.

In order to evaluate the effect of the invented compositions and formulations, the Franz cell in vitr penetration method was used on hoof membranes of bovine origin as a replacement for nails. The hoof is regarded as a fully sufficient replacement for human nails. All in vitr penetration experiments were performed in triplicate.

The penetration properties of the formulations were tested in a FDC-400 Franz cell equipment from Crown Glass Company with 9 cells with the cell orifice area 2.01 cm². The experiments are made in triplicates, unless otherwise is stated.

The hoof material was of bovine origin and was sliced to 100 um thick membranes with a microtome. The hoof membranes were hydrated for 15 minutes prior to mounting on the diffusion cells. Only membranes from the sole of the hoof were used. The membranes used were taken from the same part of the hoof to ensure similar penetration behavior of the membranes.

The receptor fluid used was citric acid buffer at pH 3.7 that was degassed for 10 minutes with helium prior to use. The cell volume was 7 ml. Sampling was performed after 6 hours.

The flux was normalized to the flux of a 1% terbinafine composition so that the results of a evaporating composition can be compared to that of a non-evaporating composition. Therefore, the flux is here described as μg of terbinafine/% tbf*h*cm² and the results from the penetration experiments has been calculated according to the equation:

Normalized flux=Δm/(Δt*A*% tbf)

### Where

Δm=mass increase of terbinafine in the receptor fluid in μg
Δt=time between observations in hours
A=membrane surface area in cm²
% tbf=the weight percentage of terbinafine in the composition.

Terbinafine was used in the form of terbinafine hydrochloride. Naftifine was used in the form of its hydrochloride

### Example 1

A terbinafine-containing composition was prepared by mixing and dissolving the following components in amounts indicated in Table 1.

TABLE 1

| | | |
|---|---|---|
| Propylene glycol | 7 | g |
| Lactic acid | 1 | g |
| Urea | 2 | g |
| Terbinafine | 1 | g |
| Ethyl acetate | 89 | g |
| Total | 100 | g |

The resulting composition appeared as a suspension of particles consisting of urea and terbinafine. After evaporation for 2 minutes in 20° C., a clear solution was formed, which shows that the particles had dissolved.

### Example 2

A terbinafine-containing composition was prepared by mixing and dissolving the following components in amounts indicated in Table 2.

TABLE

| | | |
|---|---|---|
| Propylene glycol | 3.5 | g |
| Lactic acid | 0.5 | g |
| Urea | 1 | g |
| Terbinafine | 1 | g |
| Ethyl acetate | 94 | g |
| Total | 100 | g |

The composition is a suspension of particles consisting of urea and terbinafine. After evaporation as in Example 1, a clear solution was formed.

### Example 3

The following compositions were manufactured and tested for ease of application, evaporation, feel of film and how easy the film was to wash off. In 3A cellulose acetate butyrate (CAB) was included. In 3B the methacrylate Eudragit was included.

TABLE

| Formulation | 3A | 3B |
|---|---|---|
| Ethyl acetate | 85.95 | 85.95 |
| CAB | 8.00 | 0 |
| Eudragit | 0 | 8.00 |
| EDTA | 0.05 | 0.05 |
| Terbinafine | 1.00 | 1.00 |
| Propyleneglycol | 3.50 | 3.50 |
| Urea | 1.00 | 1.00 |
| Lactic acid | 0.50 | 0.50 |

Both compositions contained a suspension of particles that cleared upon evaporation of the volatile vehicle. The two compositions were found to be equal in ease of application, time for drying, appearance and feel.

### Example 4

A terbinafine-containing composition was prepared by mixing and dissolving the following components in amounts indicated in Table 4.

TABLE

| | | |
|---|---|---|
| Propylene glycol | 7 | g |
| Lactic acid | 1 | g |
| Urea | 2 | g |
| Terbinafine | 1 | g |
| Eudragit | 4 | g |
| Ethyl acetate | 85 | g |
| Total | 100 | g |

The composition appeared as a suspension of particles consisting of urea and terbinafine. After evaporation as in Example 1, a clear film was formed.

### Example 5

The formulations 5A and 5B were manufactured and tested for stability and penetration.

TABLE

| Formulation | 5A | 5B |
|---|---|---|
| Propylene glycol | 7 g | 69 g |
| Lactic acid | 1 g | 10 g |
| Urea | 2 g | 20 g |
| Terbinafine | 1 g | 1 g |
| Ethyl acetate | 62 g | |
| Butyl acetate | 27 g | |
| Total | 100 g | 100 g |

The composition 5A was a suspension of particles consisting of urea and terbinafine, whereas 5B was a clear solution. After evaporation as in Example 1, a clear film was formed. In hoof penetration studies a flux of 77.9 μg/% tbf*h*cewas recorded for the 5A composition. This is 6.8 times higher than the flux of the 1% non-evaporating control composition 5B, (11.43 μg/% tbf*h*cm²)

### Example 6

The formulations in example 5 were subjected to stability studies. The products were stored in glass containers at 25° C. for several months The content of terbinafine and terbinafine related substances was determined by HPLC at the times indicated in table 6. The fraction of terbinafine-related substances increased substantially in the control formulation (formulation 5B), indicating the formation of degradation products of terbinafine. The amount of terbinafine decreased over time in formulation 5B. The fraction of terbinafine-related substances and the concentration of terbinafine were essentially the same over time in formulation 5A, indicating that this formulation is stable.

TABLE 6
Result of stability studies

| | | 0 months | 1 months | 3 months | 6 months |
|---|---|---|---|---|---|
| Formulation | Terbinafine | 1.03 | 1.00 | 1.05 | 1.05 |
| 5 A | (%) | | | | |
| | Related | 0.28 | 0.22 | 0.30 | 0.29 |
| | substances | | | | |
| | (% o.l.a) | | | | |
| Formulation | Terbinafine | 1.00 | 0.94 | 0.93 | n.a. |
| 5 B | (%) | | | | |
| | Related | 0.33 | 0.67 | 2.05 | n.a. |
| | substances | | | | |
| | (% o.o.l.a.) | | | | |

### Example 7

A terbinafine-containing composition was prepared by mixing and dissolving the following components in amounts indicated in Table 7:

TABLE

| Formulation | 7A | 7B |
|---|---|---|
| Propyleneglycol | 3.5 g | 69 g |
| Lactic acid | 0.5 g | 10 g |
| Urea | 1 g | 20 g |
| EDTA | 0.005 g | |
| Terbinafine | 1 g | 1 g |
| Eudragit | 4.1 g | |
| Ethyl acetate | 62.9 g | |
| Butyl acetate | 26.95 g | |
| Total | 100 g | 100 g |

The composition 7A appeared as a suspension of particles consisting of urea and terbinafine. After evaporation as in example 1 a clear film is formed. 7B was a clear solution. In hoof penetration studies a flux of 54.3 μg/% tbf*h*cm² was recorded for composition 7A. This is about 5 times higher than the flux of a 1% terbinafine control formulation, the composition 7B, (11.43 μg/% tbf*h*cm²).

### Example 8

A naftifine-containing composition was prepared by mixing and dissolving the following components in amounts indicated in Table 8:

TABLE

| | | |
|---|---|---|
| Propylene glycol | 7 | g |
| Lactic acid | 1 | g |
| Urea | 2 | g |
| Naftifine | 1 | g |
| EDTA | 0.005 | g |
| Eudragit | 4 | g |
| Ethyl acetate | 60 | g |
| Butyl acetate to total | 100 | g |

The composition appeared as a suspension of particles consisting of urea, EDTA and naftifine. After evaporation as in example 1, a clear film was formed.

### Example 9

Three formulations were manufactured and tested for penetration through human nails. The experiment was performed in a Franz cell using dedicated nail adaptors both from PermeGear Inc., USA. Nails were acquired from Sciencecare USA and Biopred France. The formulations were applied once daily and the penetration through the nail was monitored according to schedule during 20 days. The receptor fluid used was a phosphate buffer at pH 7.4 with a surfactant Brij 20 to improve solubility of terbinafine.

TABLE

| Formulation | 9A | | 9B | | Standard | |
|---|---|---|---|---|---|---|
| Propylene glycol | 7 | g | 7 | g | 69 | |
| Lactic acid | 1 | g | 1 | g | 10 | |
| Urea | 2 | g | 1 | g | 20 | |
| Terbinafine | 1 | g | 1 | g | 1 | |
| EDTA | 0.05 | g | | | | |
| MMA | 8 | g | | | | |
| Tween 80 | 0.1 | g | | | | |
| Ethyl acetate | 56.3 | g | 90 | g | | |
| Butyl acetate | 24.55 | | | | | |
| Total | 100 | g | 100 | g | 100 | g |

The two evaporating formulations in table 9 (9A and 9B) penetrated 7 to 9 times faster than the nonevaporating control formulation as measured by normalized flux. This demonstrates the superiority of the invented formulation and also that the addition of a polymer, a surfactant and EDTA did not hinder penetration of terbinafine through nail from the invented formulations. Both formulations generated nail concentrations of more than 200 μg/g after 20 days of treatment, measured by nail extraction.

### Example 10

The compositions in table 10 were tested for penetration.

TABLE 10

| Formulation | 10A | | Control |
|---|---|---|---|
| Propylene glycol | 5.6 | g | 69 |
| Lactic acid | 0.8 | g | 10 |
| Urea | 1.6 | g | 20 |
| Terbinafine | 1 | g | 1 |
| Acetylcysteine | 1 | g | |
| Ethyl acetate | 90 | g | |

The flux through hoof in the earlier described model was 3.8 higher for 10A than the control composition which lacked the volatile vehicle.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

1-42. (canceled)

43. An antifungal composition for topical application on a nail comprising:
from about 1% to about 20% of a diol component;
from about 0.1% to about 4% of an organic acid component;
from about 75% to about 96% of a volatile vehicle;
from about 0.01% to about 10% of an antifungal agent; and
from about 0.3% to about 20% of a keratolytic agent, wherein the antifungal agent and the keratolytic agent are soluble in the composition in the absence of the volatile vehicle, and that at least one in the group selected from the antifungal agent and the keratolytic agent is in the solid state in the composition in the presence of the volatile vehicle.

44. The composition according to claim 43, wherein the diol component comprises at least one diol selected from the group consisting of ethylene glycol, propylene glycol, propanediol, butyldiol, butanediol, pentanediol, hexanediol, and mixtures thereof.

45. The composition according to claim 43, wherein the organic acid component comprises at least one C₁₋₁₀ carboxylic acid.

46. The composition according to claim 45, wherein the C₁₋₁₀ carboxylic acid comprises an alpha-hydroxy carboxylic acid.

47. The composition according to claim 46, wherein the alpha-hydroxy carboxylic acid comprises at least one of lactic acid and citric acid.

48. The composition according to claim 43, wherein the antifungal agent comprises an allylamine antifungal agent.

49. The composition according to claim 48, wherein the allylamine antifungal agent is selected among terbinafine and naftifine.

50. The composition according to claim 49, wherein the allylamine antifungal agent is terbinafine.

51. The composition according to claim 43, wherein the keratolytic agent comprises at least one selected from the group consisting of urea, a sulphur containing amino acid, and mixtures thereof.

52. The composition according to claim 51, wherein the keratolytic agent comprises urea.

53. The composition according to claim 43, wherein the volatile vehicle comprises at least one compound selected from the group consisting of methyl acetate, ethyl acetate, butyl acetate, ketones (e.g. acetone, methyl ethyl ketone and methyl isobutyl ketone), ethanol, isopropanol and mixtures thereof.

54. The composition according to claim 53, wherein the volatile vehicle comprises butyl acetate.

55. The composition according to claim 53, wherein the volatile vehicle comprises ethyl acetate or a mixture of ethyl acetate and butyl acetate.

56. The composition according to claim 43, comprising from about 1% to about 20% of propylene glycol, from about 0.8% to about 1.2% of lactic acid, from about 0.8% to about 1.2% of terbinafine, from about 1.8% to about 2.2% of urea, and from about 78% to about 95% of a volatile vehicle.

57. The composition according to claim 43, consisting of: from about 6% to about 8% of propylene glycol, from about 0.8% to about 1.2% of lactic acid, from about 1.8% to about 2.2% of urea, from about 0.8% to about 1.2% of terbinafine, from about 6% to about 10% of a film forming agent, from about 54% to about 60% of ethyl acetate, from about 22% to about 26% of butyl acetate, a detergent and a sequestering agent.

58. A composition according to claim 43 for use in treating a fungal infection of a nail.

59. A method of treating a fungal infection of a nail, comprising administering to a nail of a patient an antifungal composition for topical application on a nail comprising:
from about 1% to about 20% of a diol component;
from about 0.1% to about 4% of an organic acid component;
from about 75% to about 96% of a volatile vehicle;
from about 0.01% to about 10% of an antifungal agent; and
from about 0.3% to about 20% of a keratolytic agent, wherein the antifungal agent and the keratolytic agent are soluble in the composition in the absence of the volatile vehicle, and that at least one in the group selected from the antifungal agent and the keratolytic agent is in the solid state in the composition in the presence of the volatile vehicle.

60. A method of using an antifungal composition for topical application on a nail comprising:
from about 1% to about 20% of a diol component;
from about 0.1% to about 4% of an organic acid component;
from about 75% to about 96% of a volatile vehicle;
from about 0.01% to about 10% of an antifungal agent; and
from about 0.3% to about 20% of a keratolytic agent, wherein the antifungal agent and the keratolytic agent are soluble in the composition in the absence of the volatile vehicle, and that at least one in the group selected from the antifungal agent and the keratolytic agent is in the solid state in the composition in the presence of the volatile vehicle, wherein the method comprises the step of using the composition in the manufacture of a medicament for treating a fungal infection of a nail.
